(19) United States
(12) Patent Application Publication
(10) Pub. No.: US 2024/0317680 A1
(43) Pub. Date: Sep. 26, 2024

(54) URETHANE (METH)ACRYLATE

(71) Applicant: AGC Inc., Tokyo (JP)

(72) Inventors: Makito NAKAMURA, Chiyoda-ku (JP); Chitoshi SUZUKI, Chiyoda-ku (JP)

(73) Assignee: AGC Inc., Tokyo (JP)

(21) Appl. No.: 18/735,267

(22) Filed: Jun. 6, 2024

Related U.S. Application Data
(63) Continuation of application No. PCT/JP2022/043687, filed on Nov. 28, 2022.

(30) Foreign Application Priority Data
Dec. 9, 2021 (JP) 2021-200201

(51) Int. Cl. C07C 271/22; C07C 269/02; C08G 18/10; C08G 18/32; C08G 18/67; C08G 18/75; C09J 175/04

(52) U.S. Cl. CPC C07C 271/22; C07C 269/02; C08G 18/10; C08G 18/3215; C08G 18/6755; C08G 18/755; C09J 175/04

(57) ABSTRACT

Provided is a urethane (meth)acrylate that can be cured to form a cured product excellent in stretchability, flexibility, and foldability, as well as shape recoverability when folded. The urethane (meth)acrylate of the present invention is represented by the following formula (1) and has a number average molecular weight (Mn) of 16000 to 60000, a ratio of a weight average molecular weight (Mw) to the (Mn), Mw/Mn, of 1.0 to 1.2, and a urethane group mass content of 0.18 to 0.73 mass % based on a total mass of the urethane (meth)acrylate:

R²—NHC(═O)O—R¹—OC(═O)NH—R³ (1)
wherein R¹ is a residual group formed by removing two hydroxy groups from a molecule of a diol selected from the group consisting of a polyether diol, a polyester diol, and a polycarbonate diol; and R² and R³ are each independently a residual group formed by removing an isocyanate group from a monoisocyanate having one or more (meth)acryloyloxy groups per molecule.

### TECHNICAL FIELD

The present invention relates to a urethane (meth)acrylate suitable for an adhesive and a method for producing the same, a curable composition including the urethane (meth)acrylate, and a cured product thereof.

### BACKGROUND ART

A urethane (meth)acrylate can give a functionalized polymer excellent in various characteristics, including flexibility, toughness, impact resistance, and adhesion, when it is used as a monomer, and thus, the urethane (meth)acrylate is a compound with a high versatility as a monomer. Examples of the application thereof include utilization as an ingredient of an adhesion for portions where impact resistance is required, such as a touch panel of image displaying devices and its surroundings.

A general method for synthesizing a urethane (meth)acrylate is a method including reacting a prepolymer with a compound having a hydroxy group and a (meth)acryloyloxy group (for example, 2-hydroxyethyl acrylate) and an isocyanate group-terminated prepolymer. Another synthesis method is also known that includes reacting a polyol with a compound having an isocyanate group and a (meth)acryloyloxy group.

The kind of the polyol as a starting material of the urethane (meth)acrylate obtained by any of these synthesis methods greatly affects the difference in the characteristics of cured products obtained by using the urethane (meth)acrylate as a monomer.

For example, PTL 1 discloses a (meth)acryloyl-modified polyether (urethane (meth)acrylate) obtained by reacting 2-(meth)acryloyloxyethyl isocyanate with a polyether polyol having a number average molecular weight of 3000 to 15000 used as a polyol.

### CITATION LIST

### Patent Literature

PTL 1: JP 2012-162705 A1

### SUMMARY OF INVENTION

### Technical Problem

For a device having a structure such that its face is to be folded, such as a flexible display or a foldable device, it is necessary to have shape recoverability when folded.

The urethane (meth)acrylate specifically described in PTL1 has a number average molecular weight of at most 15500. Such a urethane (meth)acrylate can give a cured product excellent in impact absorbency, but not in shape recoverability when folded.

The present invention has made under such circumstances, and an object thereof is to provide a urethane (meth)acrylate that can be cured to form a cured product excellent in stretchability, flexibility, and foldability, as well as shape recoverability when folded.

### Solution to Problem

The present invention is based on a finding that a cured product obtained by using a urethane (meth)acrylate having a structure derived from a specific diol shows a small residual strain, and has favorable shape recoverability when folded.

The present invention provides the following means.

[1] A urethane (meth)acrylate represented by the following formula (1) and having a number average molecular weight (Mn) of 16000 to 60000, a ratio of a weight average molecular weight (Mw) to the number average molecular weight (Mn), Mw/Mn, of 1.0 to 1.2, and a urethane group mass content of 0.18 to 0.73 mass % based on a total mass of the urethane (meth)acrylate:

R²—NHC(═O)O—R¹—OC(═O)NH—R³ (1)
wherein R¹ is a residual group formed by removing two hydroxy groups from a molecule of a diol selected from the group consisting of a polyether diol, a polyester diol, and a polycarbonate diol; and
R² and R³ are each independently a residual group formed by removing an isocyanate group from a monoisocyanate having one or more (meth)acryloyloxy groups per molecule.

[2] The urethane (meth)acrylate according to [1], wherein the urethane (meth)acrylate is a urethane reaction product between the diol and the monoisocyanate, and the diol has a molecular weight, in terms of hydroxyl value, of 16000 or more.

[3] The urethane (meth)acrylate according to [1] or [2], wherein the diol is a polyether diol.

[4] The urethane (meth)acrylate according to any one of claims 1 to 3, wherein the polyether diol has an oxyalkylene group as a constitutional unit, and the oxyalkylene group comprises 50 mass % or more of an oxypropylene group based on 100 mass % of all the oxyalkylene group.

[5] A method for producing a urethane (meth)acrylate, comprising obtaining a reaction product of a urethane reaction between 1 part by mole of a diol and 2 parts by mole of a monoisocyanate, wherein the urethane (meth)acrylate has a number average molecular weight (Mn) of 16000 to 60000, a ratio of a weight average molecular weight (Mw) to the number average molecular weight (Mn), Mw/Mn, of 1.0 to 1.2, and a urethane group mass content of 0.18 to 0.73 mass % based on a total mass of the urethane (meth)acrylate, and is represented by the following formula (1):

R²—NHC(═O)O—R¹—OC(═O)NH—R³ (1)
wherein R¹ is a residual group formed by removing two hydroxy groups from a molecule of a diol selected from the group consisting of a polyether diol, a polyester diol, and a polycarbonate diol; and R² and R³ are each independently a residual group formed by removing an isocyanate group from a monoisocyanate having one or more (meth)acryloyloxy groups per molecule.

[6] The method according to [5], wherein the diol has a molecular weight, in terms of hydroxyl value, of 16000 or more.

[7] The method according to [5] or [6], wherein the diol is the polyether diol.

[8] The method according to any one of claims [5] to [7], wherein the polyether diol has an oxyalkylene group as a constitutional unit, and the oxyalkylene group includes 50 mass % or more of an oxypropylene group based on 100 mass % of all the oxyalkylene group.

[9] A curable composition comprising the urethane (meth)acrylate according to any one of claims [1] to [4].

[10] The curable composition according to [9], wherein a content of the urethane (meth)acrylate in the curable composition is 50 mass % or more.

[11] The curable composition according to [9] or [10], wherein the curable composition is an adhesive.

[12] A cured product obtained by curing the curable composition according to any of claims [9] to [11].

[13] An article comprising the cured product according to [12].

### Advantageous Effects of Invention

According to the present invention, there can be provided a urethane (meth)acrylate that can be cured to form a cured product excellent in stretchability, flexibility, and foldability, as well as shape recoverability when folded.

Accordingly, the urethane (meth)acrylate of the present invention is useful for application such as an adhesive, a middle coat of coating, an ink binder, etc. for a foldable portion in a flexible display, a foldable device, or the like.

### DESCRIPTION OF EMBODIMENTS

Definitions and meanings of terms and notation herein are shown below.

“(Meth)acryloyloxy” is a collective term for acryloyloxy and methacryloyloxy. Similarly, “(meth)acryl” is a collective term for acryl and methacryl, and “(meth)acrylate” is a collective term for acrylate and methacrylate.

A number average molecular weight (Mn) and a weight average molecular weight (Mw) are each a molecular weight, in terms of polystyrene, as measured through measurement by gel permeation chromatography (GPC) based on a calibration curve prepared by using a standard polystyrene sample.

A urethane bond concentration is a proportion of the mass of the urethane groups in the total mass of the urethane (meth)acrylate, and is a value calculated by the formula: x×59/(mass of urethane (meth)acrylate)×100, wherein it is considered that the whole amount (x mole) of the isocyanate groups of an isocyanate compound as a reactant material has formed urethane bonds (formula weight 59).

The “molecular weight in terms of hydroxyl value” is a value calculated from the formula: 56100×(the number of hydroxyl groups per molecule)/(hydroxyl value [mg KOH/g]). The hydroxyl value can be measured by measurement in accordance with JIS K 1557:2007.

A viscosity is a value as measured at 25° C. using an E-type viscometer.

An “NCO index” is a value, in percent, of an equivalent ratio of the isocyanate group of an isocyanate compound to the hydroxyl group of a polyol.

[Urethane (meth)acrylate]

The urethane (meth)acrylate of the present invention is represented by the following formula (1) and has an Mn of 16000 to 60000, a ratio of the Mw to the Mn, Mw/Mn, of 1.0 to 1.2, and a urethane bond concentration of 0.18 to 0.73 mass %:

R²—NHC(═O)O—R¹—OC(═O)NH—R³ (1)

wherein R¹ is a residual group formed by removing two hydroxy groups from a molecule of a diol selected from the group consisting of a polyether diol, a polyester diol, and a polycarbonate diol; and R² and R³ are each independently a residual group formed by removing an isocyanate group from a monoisocyanate having one or more (meth)acryloyloxy groups per molecule.

Such a urethane (meth)acrylate has a skeleton derived from a diol having a larger molecular weight, and has a sharp molecular weight distribution, and accordingly, it is considered that there is a tendency that a cured product obtained by using it as a monomer has a uniform crosslinking network formed therein. Thus, the urethane (meth)acrylate can form a cured product having a large tensile elongation, a small storage shearing elastic modulus, and a small residual strain, and being excellent in stretchability, flexibility, and foldability, as well as shape recoverability when folded.

The urethane (meth)acrylate of the present invention is the compound represented by formula (1) described above.

The urethane (meth)acrylate is preferably a urethane acrylate in view of rapid polymerization.

### (R¹)

In formula (1), R¹ is a residual group formed by removing two hydroxy groups from a molecule of a diol selected from the group consisting of a polyether diol, a polyester diol, and a polycarbonate diol.

The diol is preferably a polyether diol in view of shape recoverability, when folded, of a cured product obtained from the urethane (meth)acrylate.

### <Polyether Diol>

The polyether diol is preferably a polymer having two hydroxy groups and also having an oxyalkylene group as a constitutional unit. The polyether diol can be obtained by ring-opening polymerization of a compound having a cyclic ether structure with an initiator having two active hydrogen atoms. A commercially available product can also be used.

The oxyalkylene group preferably contains a linear or branched alkylene group having 1 to 14 carbon atoms, and more preferably 2 to 4 carbon atoms. A single kind of an oxyalkylene group may be included, or two or more kinds of oxyalkylene groups may be included.

Specifically, the oxyalkylene group is preferably one or more selected from the group consisting of an oxyethylene group, an oxypropylene group, and an oxytetramethylene group, and more preferably includes an oxypropylene group. In view of easily obtaining a cured product of the urethane (meth)acrylate having favorable flexibility, the oxyalkylene group preferably includes 50 mass % or more, more preferably 60 to 100 mass %, even more preferably 80 to 100 mass %, further more preferably 100 mass % of the oxypropylene group, based on 100 mass % of all the oxyalkylene group.

It is considered that the content of the oxypropylene group in all the oxyalkylene group corresponds to the amount, in parts by mass, of propylene oxide blended per total 100 parts by mass of starting materials serving as the sources the oxyalkylene group when synthesizing the polyether diol.

Examples of the compound having a cyclic ether structure include ethylene oxide, propylene oxide, 1,2-butylene oxide. 2,3-butylene oxide, methyl glycidyl ether, butyl glycidyl ether. 2-ethylhexyl glycidyl ether, lauryl glycidyl ether, hexyl glycidyl ether, and tetrahydrofuran. Among these, ethylene oxide and propylene oxide are preferable.

Examples of a group having the active hydrogen atoms in the initiator include a hydroxyl group, a carboxy group, an amino group having a hydrogen atom bonded to a nitrogen atom. Among these, a hydroxyl group is preferable, and an alcoholic hydroxyl group is more preferable.

Examples of the initiator having two active hydrogen atoms include a glycol such as ethylene glycol, propylene glycol, diethylene glycol, dipropylene glycol, butylene glycol, 1,4-butanediol, 1,6-hexanediol, triethylene glycol, tripropylene glycol, a polyoxyalkylenediol (e.g., polyethylene glycol and polypropylene glycol); a bisphenol such as bisphenol A, bisphenol F, and bisphenol AD; a dihydroxybenzene such as catechol, resorcinol, and hydroquinone; a primary amine such as methylamine, ethylamine, propylamine, and butylamine. Among these, the glycol is preferable.

The ring-opening polymerization can be carried out with a known catalyst, including an alkali catalyst such as potassium hydroxide, a transition metal compound/porphyrin complex catalyst such as a complex obtained by reacting an organoaluminum compound with porphyrin, a double metal cyanide complex catalyst, and a catalyst consisting of a phosphazene compound. Among these catalysts, the double metal cyanide complex (DMC) catalyst is preferable in view of obtaining a polyether diol having a sharp molecular weight distribution. The composite metal cyanide complex catalyst used may be a known compound, and examples thereof include zinc hexacyanocobaltate complex having with tert-butanol as a ligand.

The production of the polyether diol by ring-opening polymerization with the DMC catalyst may be carried out by a known method, and production methods that can be employed are disclosed in WO 2003/062301, WO 2004/067633, JP 2004-269776 A1, JP 2005-15786 A1, WO 2013/065802, and JP 2015-10162 A1, for example.

### <Polyester Diol>

The polyester diol used may be, for example, one obtained by a known production method including polycondensation between a dibasic acid and a glycol, or ring-opening polymerization of a cyclic ester such as &-caprolactone. A commercially available product may also be used.

Examples of the dibasic acid include an aliphatic dibasic acid, such as succinic acid, adipic acid, maleic acid, and fumaric acid: an alicyclic dibasic acid, such as 1,4-cyclohexanedicarboxylic acid; an aromatic dibasic acid, such as phthalic acid, isophthalic acid, and terephthalic acid; and an acid anhydride thereof. The dibasic acids may be used singly or in combinations of two or more thereof.

Examples of the glycol include ethylene glycol, propylene glycol, diethylene glycol, dipropylene glycol, butylene glycol, 1,4-butanediol, 1,6-hexanediol, triethylene glycol, tripropylene glycol, and polyoxyalkylene glycol. The glycols may be used singly or in combinations of two or more thereof.

### <Polycarbonate Diol>

The polycarbonate diol used may be, for example, one obtained by a known production method including polycondensation between a glycol and a carbonate compound. A commercially available product may also be used.

Specific examples of the glycol include the same glycols as listed above for using production of the polyester diol. The glycols may be used singly or in combinations of two or more thereof.

Examples of the carbonate compound include dimethyl carbonate, diethyl carbonate, dipropyl carbonate, dibutyl carbonate, diphenyl carbonate, ethylene carbonate, trimethylene carbonate, propylene carbonate, 1,2-butylene carbonate, and neopentylene carbonate. The carbonate compounds may be used singly or in combinations of two or more thereof.

(R² and R³)

In formula (1), R² and R³ are each independently a residual group formed by removing an isocyanate group from a monoisocyanate having one or more (meth)acryloyloxy groups per molecule.

R² and R³ may be the same or different from each other. R² and R³ are preferably the same in view of efficiently synthesizing the urethane (meth)acrylate.

The monoisocyanate having a (meth)acryloyloxy groups is preferably a compound having a hydrocarbon skeleton with an isocyanate group and also one or more (meth)acryloyloxy groups bonded to the hydrocarbon skeleton. The number of the (meth)acryloyloxy groups may be one or more.

The hydrocarbon skeleton is preferably an aliphatic hydrocarbon group or an alicyclic hydrocarbon group. The aliphatic hydrocarbon group or alicyclic hydrocarbon group preferably has 8 or less carbon atoms, and more preferably 2 to 6 and even more preferably 2 to 4 carbon atoms.

Examples of the monoisocyanate having a (meth)acryloyloxy groups include a compound having one (meth)acryloyloxy group such as isocyanatomethyl (meth)acrylate and 2-isocyanatoethyl (meth)acrylate; a compound having two (meth)acryloyloxy groups such as 1,1-(bis(meth)acryloyloxymethyl)ethyl isocyanate and 1,1-(bis(meth)acryloyloxymethyl)propyl isocyanate. Examples of a commercially available product thereof include “Karenz (registered trademark; hereinafter, omitting the indication) AOI” (2-isocyanate ethyl acrylate), “Karenz MOI” (2-isocyanate ethyl methacrylate), and “Karenz BEI” (1,1-(bis acryloyloxymethyl)ethyl isocyanate) (all manufactured by Showa Denko K.K.).

### (Number Average Molecular Weight)

The urethane (meth)acrylate has an Mn of 16000 to 60000, preferably 17000 to 55000, and more preferably 18000 to 50000.

When the Mn is within the above-described range, a cured product of the urethane (meth)acrylate shows favorable stretchability and is also excellent in shape recoverability when folded.

The Mw/Mn is 1.0 to 1.2, preferably 1.02 to 1.17, and more preferably 1.05 to 1.15.

The Mw/Mn is a measure that indicates a degree of dispersion (broadness) of the molecular weight distribution. A ratio Mw/Mn of 1 indicates monodispersity, and a ratio Mw/Mn closer to 1 means a sharper molecular weight distribution.

When the molecular weight distribution is within the above-described range, the urethane (meth)acrylate has a relatively small viscosity so that a curable composition including thereof is easy to handle in working such as mixing even though the urethane (meth)acrylate has a molecular weight Mn as large as 16000 or more. Also, a cured product of the urethane (meth)acrylate has a small residual strain and tends to have a uniform crosslinking network formed therein, whereby the cured product is excellent in shape recoverability when folded.

### (Urethane Bond Concentration)

The urethane (meth)acrylate has a urethane bond concentration of 0.18 to 0.73 mass %, preferably 0.40 to 0.72 mass %, and more preferably 0.55 to 0.70 mass %.

When the urethane bond concentration is within the range described above, a cured product of the urethane (meth)acrylate shows favorable stretchability and is also excellent in shape recoverability when folded.

### (Viscosity)

In view of ease in handling, the urethane (meth)acrylate is a liquid at room temperature (25° C.), and preferably has a viscosity at 25° C. of 50 Pads or less, more preferably 40 Pa·s or less, and even more preferably 30 Pas or less.

### (Urethane Reaction)

The urethane (meth)acrylate of the present invention is obtained as a reaction product of urethane reaction between 1 part by mole of a diol and 2 parts by mole of a monoisocyanate. In other words, the urethane (meth)acrylate is a reaction product between a diol derived from R¹ in formula (1) and a monoisocyanate derived from R² and R³.

The diol preferably has a molecular weight, in terms of hydroxyl value, of 16000 or more, more preferably 16500 to 60000, and even more preferably 17000 to 55000, in view of obtaining an Mn of the urethane (meth)acrylate within the above-described range.

The urethane reaction can be carried out by a known method, and generally, the diol and the monoisocyanate are mixed with each other, and subjected to the urethane reaction with a catalyst for urethane reaction in a nitrogen gas or inert gas atmosphere.

Examples of the catalyst for urethane reaction include an organotin compound such as dibutyl tin dilaurate, dioctyl tin dilaurate, dibutyl tin dioctoate, and tin 2-ethylhexanoate; an iron compound such as iron acetylacetonate, ferric chloride; a lead compound such as lead 2-ethyl hexanoate; a bismuth compound such as bismuth 2-ethyl hexanoate; and a tertiary amine such as triethylamine and triethylenediamine. Among these, the organotin compound, lead 2-ethylhexanoate, and bismuth 2-ethyl hexanoate are preferable. The catalysts for urethane reaction may be used singly or in combinations of two or more thereof.

The amount of the catalyst used for urethane reaction is preferably 0.001 to 1 part by mass, more preferably 0.002 to 0.5 part by mass, and even more preferably 0.005 to 0.1 part by mass, per 100 parts by mass of the diol as a reactant.

The reaction temperature of the urethane reaction is preferably 20 to 100° C., more preferably 30 to 90° C., and even more preferably 40 to 80° C.

### [Curable Composition]

The curable composition of the present invention includes the urethane (meth)acrylate of the present invention described above.

In the curable composition, a polymerization initiator and other components, if necessary, are preferably contained.

The content of the urethane (meth)acrylate in the curable composition is preferably 50 mass % or more, more preferably 70 mass % or more and 100 mass % or less, and even more preferably 80 mass % or more and 100 mass % or less, in view of obtaining a cured product excellent in shape recoverability when folded.

It is preferable to uniformly mix the components to be contained in the curable composition, and for example, they can be mixed using a known mixer such as a planetary agitator/deaerator, a homogenizer, a planetary mixer, a three-roll mill, and a bead mill. The components to be contained may be mixed all at once, or mixed while added successively.

### (Polymerization Initiator)

The polymerization initiator is preferably a radical polymerization initiator, which may be a photo polymerization initiator or a thermal polymerization initiator, and any known initiator can be used.

In view of ease in controlling the polymerization reaction, the photo polymerization initiator is preferably one rendered usable by irradiation with ultraviolet rays having a wavelength of 380 nm or less, and the thermal polymerization initiator is preferably one rendered usable by heating at a temperature within a range from 50 to 120° C.

Examples of the photo polymerization initiator include 1-hydroxycyclohexylphenyl ketone, 2-hydroxy-2-methylpropiophenone, diethoxyacetophenone, 1-(4-isopropylphenyl)-2-hydroxy-2-methylpropanone, 1-(4-dodecylphenyl)-2-hydroxy-2-methylpropanone, 4-(2-hydroxyethoxy)-phenyl(2-hydroxy-2-propyl)ketone, 2-methyl-1-[4-(methylthio)phenyl]-2-morpholinopropanone, benzoin, benzoin methyl ether, benzoin ethyl ether, benzoin isopropyl ether, benzoin n-butyl ether, benzoin phenyl ether, benzyl dimethyl ketal, benzophenone, benzoyl benzoate, methyl benzoyl benzoate, 4-phenylbenzophenone-4-methoxybenzophenone, thioxanthone, 2-chlorothioxanthone, 2-methylthioxanthone, 2,4-dimethylthioxanthone, isopropylthioxanthone, 2,4-dichlorothioxanthone, 2,4-diethylthioxanthone, 2,4-diisopropylthioxanthone, 2,4,6-trimethylbenzoyl diphenylphosphine oxide, phenylbis(2,4,6-trimethylbenzoyl)phosphine oxide, methylphenyl glyoxylate, benzyl, and camphorquinone. The photo polymerization initiators may be used singly or in combinations of two or more thereof.

Examples of the thermal polymerization include an azo compound; and an organic peroxide such as hydroperoxide, dialkyl peroxide, peroxyester, diacyl peroxide, peroxydicarbonate, peroxiketal, and ketone peroxide. Specific examples include azobisisobutyronitrile, benzoyl peroxide, tert-butyl peroxy-2-ethylhexanoate, 2,5-dimethyl-2,5-di(2-ethylhexanoyl)peroxyhexane, tert-butyl peroxybenzoate, tert-butyl peroxide, cumene hydroperoxide, dicumylperoxide, di-t-butylperoxide, 2,5-dimethyl-2,5-dibutylperoxyhexane, 2,4-dichlorobenzoyl peroxide, 1,4-di(2-t-butylperoxyisopropyl)benzene, 1,1-bis(tert-butylperoxy)-3,3,5-trimethylcyclohexane, methyl ethyl ketone peroxide, and 1,1,3,3-tetramethyl butyl peroxy-2-ethyl hexanoate. The thermal polymerization initiators may be used singly or in combinations of two or more thereof.

The content of the polymerization initiator in the curable composition is preferably 0.001 to 20 parts by mass, more preferably 0.01 to 10 parts by mass, and even more preferably 0.1 to 7 parts by mass, per 100 parts by mass of the urethane (meth)acrylate, in view of the moderate rate of the polymerization.

In a case where the curable composition is irradiated with light to obtain a cured product, the light source can be appropriately selected according to the light absorptivity of the photo polymerization initiator contained. Examples of the light source that can be used include a UV light-emitting diode (LED), a low-pressure mercury lamp, a high-pressure mercury lamp, a mercury xenon lamp, a metal halide lamp, a tungsten lamp, an arc lamp, an excimer lamp, an excimer laser, a semiconductor laser, a YAG laser, a laser systems including combinations of a laser and a non-linear optical crystal, and a high frequency-induced, UV light-generating device. The cumulative dose of light may be about 0.01 to 50 J/cm², for example.

After the irradiation with light, heat treatment may further be carried out, in view of stabilization of the physical properties of the cured product. Generally, the heating temperature is about 40 to 200° C., and the heating time is about 1 minute to 15 hours. The physical properties of the cured product can also be stabilized by still standing it at room temperature (about 15 to 25° C.) for about 1 to 48 hours.

In a case where the curable composition is subjected to heat treatment to obtain a cured product, the heating temperature is about 40 to 250° C. and the heating time is about 5 minutes to 24 hours, generally. Preferably, the heating time is shorter when the heating temperature is higher, and the heating time is longer when the heating temperature is lower.

### (Other Components)

The curable composition may include other components, in addition to the urethane (meth)acrylate and the polymerization initiator, in view of ease in handling and according to its application. Examples of the other components include a monomer component other than the urethane (meth)acrylate of the present invention, a catalyst, a colorant such as a pigment and a dye, a silane coupling agent, a tackifier resin, an antioxidant, a light stabilizer, a metal deactivator, an anti-rust agent, an anti-aging agent, a moisture absorbent, a hydrolysis inhibitor, a defoaming agent, and a filler. A solvent may also be included. These other components in the curable composition can be blended in the content range such that the effect of the present invention is not impaired.

The other monomer component is a compound that is copolymerizable with the urethane (meth)acrylate, and examples thereof include a (meth)acrylate such as a urethane (meth)acrylate other than the urethane (meth)acrylate of the present invention, an alkyl (meth)acrylate, a hydroxyl-containing (meth)acrylate, and an amino-containing (meth)acrylate. The other monomer components may be used singly or in combinations of two or more thereof.

The curable composition including the urethane (meth)acrylate of the present invention is suitable for application to, for example, an adhesive, a middle coat of coating, or an ink binder, and particularly suitable for an adhesive for a foldable portion in a flexible display, a foldable device, or the like since the cured product of the curable composition is excellent in shape recoverability when folded.

### [Cured Product]

The cured product of the present invention is obtained by curing the curable composition including the urethane (meth)acrylate described above. The cured product has a large tensile elongation, a small storage shearing elastic modulus, and a small residual strain, and is excellent in stretchability, flexibility, and foldability as well as shape recoverability when folded.

Therefore, these characteristics are exhibited in the applications as described above according to the present invention, and thus, there can be suitably provided various articles having the cured product of the present invention, such as s coated article, s print product, and an adhered product, and particularly articles having a foldable portion, such as a flexible display and a foldable device.

### EXAMPLES

The present invention will now be specifically described by way of Examples; however, the present invention is not limited to Examples below and various changes can be made without departing from the spirit of the present invention.

### [Compounds as Starting Materials]

Details of the various compounds used as starting materials in Examples were as follows.
DMC-TBA: Zinc hexacyanocobaltate-tert-butanol complex
PPG (1): polypropylene glycol; “EXCENOL (registered trademark) 1020,” manufactured by AGC Inc.; hydroxyl value 112.2 mg KOH/g; molecular weight, in terms of hydroxyl value, 1000)
AOI: 2-acryloyloxyethyl isocyanate; “Karenz (registered trademark) AOI,” manufactured by Showa Denko K.K.
Isophorone diisocyanate: “Desmodur (registered trademark) I,” manufactured by Sumika Covestro Urethane Co., Ltd.
2-Hydroxyethyl acrylate, manufactured by Tokyo Chemical Industry Co., Ltd.
2,5-Di-tert-butylhydroquinone, manufactured by Tokyo Chemical Industry Co., Ltd., antioxidant
PPG (2): polypropylene glycol; “EXCENOL (registered trademark) 2020,” manufactured by AGC Inc.; hydroxyl value 56.1 mg KOH/g; molecular weight, in terms of hydroxyl value, 2000
Photo polymerization initiator: phenylbis(2,4,6-trimethylbenzoyl)phosphine oxide; “Irgacure (registered trademark) 819,” manufactured by BASF SE

### [Measurement Methods]

The methods for determining various characteristics in Synthesis Examples and Production Examples described later were as follows.

### (Hydroxyl Value)

The hydroxyl value was measured in accordance with B method (automated potentiometric titration) in JIS K1557-1: 2007.

### (Molecular Weight in Terms of Hydroxyl Value)

The molecular weight in terms of hydroxyl value was calculated by the following equation from the found hydroxyl value (unit: mg KOH/g) measured as above (molecular weight of potassium hydroxide 56.1).

Molecular weight in terms of hydroxyl value=56.1×1000×(number of hydroxyl groups per molecule)/(hydroxyl value)

As the number of hydroxyl groups per molecule, the number of hydroxyl groups per molecule of the initiator used for the synthesis of the diol was used.

### (Number Average Molecular Weight (Mn) and Weight Average Molecular Weight (Mw))

The Mn and Mw of urethane acrylate were measured (in terms of polystyrene) by gel permeation chromatography (GPC) in the following conditions for measurement, and the molecular weight distribution (Mw/Mn) was calculated from these values.

### <Conditions for Measurement>

Apparatus used: “HLC-8320GPC,” manufactured by Tosoh Corporation
Column used: The following two types of columns were connected in series.

“TSKgel (registered trademark) SuperHM-H,” manufactured by Tosoh Corporation, two columns

“TSKgel (registered trademark) SuperH2000,” manufactured by Tosoh Corporation, one column
Column temperature: 40° C.
Detector: differential refractive index (RI) detector
Eluent: tetrahydrofuran
Flow rate: 0.8 mL/min
Concentration of sample: 0.5 mass %
Volume of sample injected: 100 μL
Standard sample: Polystyrene

### (Urethane Bond Concentration)

It was considered that the whole amount (x mole) of the isocyanate group of the isocyanate compound as a reactant material formed urethane bonds (formula weight 59), and the urethane bond concentration of the urethane acrylate was calculated by the following equation.

(Urethane bond concentration [mass %])=x×59/(mass of urethane acrylate)×100

### (Content of Isocyanate Groups)

The content of isocyanate groups (NCO) was quantitatively measured by reverse titration through indicator titration using a method in accordance with A method in JIS K 1603-1: 2007.

### [Synthesis of Diols]

### Synthesis Example 1

In a pressure-resistant reactor equipped with a stirrer and a nitrogen introducing tube, 0.2 g of DMC-TBA and 400 g of PPG (1) as an initiator were charged, and 7200 g of propylene oxide (hereinafter abbreviated to as “PO”) was charged at a constant rate over 7 hours in nitrogen gas atmosphere at 130° C. It was checked that the decrease of the inner pressure ceased in the pressure-resistant reactor, and diol (1) was thus obtained (PPG: hydroxyl value 6.4 mg KOH/g, molecular weight in terms of hydroxyl value 17500)

### Synthesis Example 2

Diol (2) (PPG: hydroxyl value 7.5 mg KOH/g, molecular weight in terms of hydroxyl value 15000) was obtained in the same manner as in Synthesis Example 1, except that the amount of PO added in Synthesis Example 1 was changed to 6020 g.

### Synthesis Example 3

Diol (3) (PPG: hydroxyl value 11.2 mg KOH/g, molecular weight in terms of hydroxyl value 10000) was obtained in the same manner as in Synthesis Example 1, except that the amount of PO added in Synthesis Example 1 was changed to 3880 g.

### [Production of Urethane Acrylate]

### Production Example 1

In a reactor equipped with a stirrer and a nitrogen introducing tube, 200 g of diol (1) and 3.22 g of AOI (NCO index 100) were charged, and reacted in the presence of 16 mg of bismuth 2-ethylhexanoate (0.008 parts by mass per 100 parts by mass of diol (1)) at 70° C. for 3 hours. Then 60 mg of 2,5-di-tert-butylhydroquinone (0.03 ppm by mass per 100 parts by mass of diol (1)) was added thereto to obtain urethane acrylate (1).

### Production Examples 2

Urethane acrylates (2) were obtained in the same manner as in Production Example 1, except that diol (1) used in Production Example 1 was replaced with diols (2).

### Production Example 3

In a reactor equipped with a stirrer and a nitrogen introducing tube, 200 g (0.02 mol) of diol (7) and 8.9 g (0.04 mol) of isophorone diisocyanate were charged, and reacted in the presence of 16 mg of bismuth 2-ethylhexanoate (0.008 parts by mass per 100 parts by mass of diol (3)) at 80° C. After it was checked that the content of isocyanate groups reached the theoretical value (0.81 mass %), 4.6 g (0.04 mol) of 2-hydroxyethyl acrylate was added thereto, and the reaction was carried out until the NCO content reached 0 mass %. Then, 60 mg of 2,5-di-tert-butylhydroquinone (0.03 ppm by mass per 100 parts by mass of diol (3)) was added thereto to obtain urethane acrylate (3).

### Production Example 4

Urethane acrylate (4) was obtained in the same manner as in Production Example 3, except that diol (3) used in Production Example 3 was replaced with PPG (2).

The Mn, the Mw/Mn, and the urethane bond concentration of each of urethane acrylates (1) to (4) are collectively shown in Table 1 below.

[Production of curable composition and cured product thereof]

### Examples 1 to 4

By using urethane acrylates (1) to (4) obtained in Production Examples 1 to 4, respectively, curable compositions and cured products thereof were produced, and measurement and evaluation for the items below were carried out.

The curable composition was prepared by mixing 100 parts by mass of a urethane acrylate with 0.3 parts by mass of a photo polymerization initiator.

Results of the measurement and evaluation are shown in Table 1. Example 1 is an inventive example, and Examples 2 to 4 are comparative examples.

### [Items of Evaluation]

### (Viscosity)

The viscosity of each of the urethane acrylates was measured using an E-type viscometer (“RE85U” manufactured by TOKISANGYO, 25° C.).

When the viscosity is 30 Pa-s or less, the urethane acrylates is easy to handle in production of a curable composition and a cured product thereof. A case where the viscosity was 30 Pas or less was graded as “A”, and a case where the viscosity was more than 30 Pa·s was graded as “B”. The results of the grade are shown in Table 1.

### (Tensile Elongation)

The curable composition was applied to the releasable surface of a PET film that had been silicone release-treated, to a thickness of 100 μm with an applicator. Then, the curable composition was cured using a conveyer-type UV irradiation system (manufactured by ORC MANUFACTURING CO., LTD.; mercury xenon lamp, intensity of illumination 100 mW/cm², cumulative dose of light 3 J/cm²) to prepare a test sample for tensile test.

The tensile test was conducted on the test sample in accordance with JIS K 7311: 1995 using a tension testing machine (TENSILON universal testing machine “RTG-1310,” manufactured by A&D COMPANY. Limited; tension rate 300 mm/min) to determine the tensile elongation.

As the tensile elongation is larger, the cured product has a more favorable stretchability. A case where the tensile elongation was 250% or more was graded as “A”, and a case where the tensile elongation was less than 250% was graded as “B”. The results of the grade are shown in Table 1.

### (Storage Shearing Elastic Modulus)

The curable composition was filled and hold in a 0.2-mm gap between a soda-lime glass made stage and a measurement spindle (“disposable plate D-PP20/AL/S07.” manufactured by Anton Paar GmbH). The curable composition was irradiated with UV for 300 seconds in nitrogen gas atmosphere at 35° C. using a mercury xenon lamp (“Spot-Cure (registered trademark) SP-9,” manufactured by Ushio Inc., intensity of illumination 100 mW/cm²) placed under the stage, to thereby obtain a cured product sample of the curable composition. While the curable composition was cured, the position of the spindle was follow-up controlled so as not to generate a stress in the normal direction of the spindle.

While the cured product sample was irradiated with UV, the storage shearing elastic modulus of the cured product sample was measured using a rheometer (“Physica MCR301,” manufactured by Anton Paar GmbH, 1% dynamic shear strain applied).

It can be said that as the storage shearing elastic modulus is smaller, the cured product is more flexible and has therefore more favorable foldability. A case where the storage shearing elastic modulus of the cured product was 700 kPa or less was graded as “A”, and a case where the storage shearing elastic modulus was more than 700 kPa was graded as “B”. The results of the grade are shown in Table 1.

### (Residual Strain)

To a cured product sample prepared in the same manner as for that for measuring the storage shearing elastic modulus, a 2% dynamic shear strain was applied for 30 minutes, and then the strain was released. The residual strain 30 minutes after releasing the strain was measured using a rheometer (“Physica MCR301,” manufactured by Anton Paar GmbH). The residual strain was expressed relatively to that before applying the dynamic shear strain, which was regarded as 0% strain (reference).

It can be said that the cured product has favorable shape recoverability even after releasing external force against the cured product. A case where the residual strain of the cured product was 0.1% or less was graded as “A”, and a case where the residual strain was more than 0.1% was graded as “B”. The results of the grade are shown in Table 1.

TABLE 1

| | | Example | | | |
|---|---|---|---|---|---|
| | | 1 | 2 | 3 | 4 |
| Urethane acrylate | | (1) | (2) | (3) | (4) |
| Mn | | 24600 | 19500 | 18900 | 4600 |
| Mw/Mn | | 1.12 | 1.11 | 1.34 | 1.32 |
| Urethane bond concentration | [mass %] | 0.66 | 0.77 | 2.26 | 8.82 |
| Viscosity | [Pa · s] | 25 | 16 | 58 | 25 |
| | | A | A | B | A |
| Cured product | | | | | |
| Tensile elongation | [%] | 260 | 230 | 200 | 80 |
| | | A | B | B | B |
| Storage shearing elastic | [kPa] | 640 | 355 | 819 | 800 |
| modulus | | A | A | B | B |
| Residual strain | [%] | −0.003 | 0.02 | −0.02 | 0.17 |
| | | A | A | A | B |

As can be seen from the evaluation results shown in Table 1, the urethane acrylates of the present invention (Example 1) each had a low viscosity, and therefore favorable handling workability. Also, the cured product of the curable composition including the urethane acrylate of the present invention (Example 1) had a large tensile elongation, a small storage shearing elastic modulus, and a small residual strain, and it can thus be said that the cured product is excellent in stretchability, flexibility, and foldability, as well as shape recoverability when folded.

1. A urethane (meth)acrylate represented by the following formula (1) and having a number average molecular weight (Mn) of 16000 to 60000, a ratio of a weight average molecular weight (Mw) to the number average molecular weight (Mn), Mw/Mn, of 1.0 to 1.2, and a urethane group mass content of 0.18 to 0.73 mass % based on a total mass of the urethane (meth)acrylate:
R²—NHC(═O)O—R¹—OC(═O)NH—R³ (1)
wherein R¹ is a residual group formed by removing two hydroxy groups from a molecule of a diol selected from the group consisting of a polyether diol, a polyester diol, and a polycarbonate diol; and
R² and R³ are each independently a residual group formed by removing an isocyanate group from a monoisocyanate having one or more (meth)acryloyloxy groups per molecule.

2. The urethane (meth)acrylate according to claim 1, wherein the urethane (meth)acrylate is a urethane reaction product between the diol and the monoisocyanate, and the diol has a molecular weight, in terms of hydroxyl value, of 16000 or more.

3. The urethane (meth)acrylate according to claim 1, wherein the diol is a polyether diol.

4. The urethane (meth)acrylate according to claim 1, wherein the polyether diol has an oxyalkylene group as a constitutional unit, and the oxyalkylene group comprises 50 mass % or more of an oxypropylene group based on 100 mass % of all the oxyalkylene group.

5. A method for producing a urethane (meth)acrylate, comprising obtaining a reaction product of a urethane reaction between 1 part by mole of a diol and 2 parts by mole of a monoisocyanate,
wherein the urethane (meth)acrylate has a number average molecular weight (Mn) of 16000 to 60000, a ratio of a weight average molecular weight (Mw) to the number average molecular weight (Mn), Mw/Mn, of 1.0 to 1.2, and a urethane group mass content of 0.18 to 0.73 mass % based on a total mass of the urethane (meth)acrylate, and is represented by the following formula (1):
R²—NHC(═O)O—R¹—OC(═O)NH—R³ (1)
wherein R¹ is a residual group formed by removing two hydroxy groups from a molecule of a diol selected from the group consisting of a polyether diol, a polyester diol, and a polycarbonate diol; and
R² and R³ are each independently a residual group formed by removing an isocyanate group from a monoisocyanate having one or more (meth)acryloyloxy groups per molecule.

6. The method according to claim 5, wherein the diol has a molecular weight, in terms of hydroxyl value, of 16000 or more.

7. The method according to claim 5, wherein the diol is a polyether diol.

8. The method according to claim 5, wherein the polyether diol has an oxyalkylene group as a constitutional unit, and the oxyalkylene group comprises 50 mass % or more of an oxypropylene group based on 100 mass % of all the oxyalkylene group.

9. A curable composition comprising the urethane (meth)acrylate according to claim 1.

10. The curable composition according to claim 9, wherein a content of the urethane (meth)acrylate in the curable composition is 50 mass % or more.

11. The curable composition according to claim 9, wherein the curable composition is an adhesive.

12. A cured product obtained by curing the curable composition according to claim 9.

13. An article comprising the cured product according to claim 12.
